(19) United States
(12) Patent Application Publication
(10) Pub. No.: US 2023/0233814 A1
(43) Pub. Date: Jul. 27, 2023

(54) Rapidly Insertable Central Catheter Insertion Assemblies and Methods of Sheath Removal

(71) Applicant: Bard Access Systems, Inc., Salt Lake City, UT (US)

(72) Inventors: Glade H. Howell, Draper, UT (US); Daniel B. Blanchard, Bountiful, UT (US); Eric W. Lindekugel, Salt Lake City, UT (US); Kyle G. Thornley, Farmington, UT (US)

(21) Appl. No.: 18/099,185

(22) Filed: Jan. 19, 2023

Related U.S. Application Data
(60) Provisional application No. 63/302,860, filed on Jan. 25, 2022.

(51) Int. Cl. A61M 25/09; A61M 25/06

(52) U.S. Cl. CPC A61M 25/09; A61M 25/0668; A61M 25/0606

(57) ABSTRACT

A rapidly insertable central catheter (“RICC”) insertion assembly can include a RICC, an introducer needle, a coupler coupling the RICC and the introducer needle together, and a sheath-splitting means for splitting a sheath body of the introducer needle away from an access guidewire. The sheath-splitting means allows the access guidewire to escape from the sheath body and remain in a needle tract established with the introduce needle. The splitting of the sheath body by the sheath-splitting means can be initiated by withdrawing the sheath body by a pair of finger tabs extending from a sheath hub around the proximal portion of the sheath body, twisting the introducer needle or a needle hub around the proximal portion of the needle shaft, triggering one or more triggers disposed in the coupler housing, or pushing a catheter tip of the RICC into the sheath body.

### PRIORITY

This application claims the benefit of priority to U.S. Provisional Application No. 63/302,860, filed Jan. 25, 2022, which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are rapidly insertable central catheter (“RICC”) insertion assemblies and methods of sheath removal that address the foregoing.

### SUMMARY

Disclosed herein is a RICC insertion assembly including, in some embodiments, a RICC, an introducer needle, and a coupler coupling the RICC and the introducer needle together. The introducer needle includes a needle shaft and a sheath body over the needle shaft. The needle shaft includes a longitudinal needle slot extending from a proximal portion of the needle shaft through a distal needle tip. The sheath body seals the needle slot thereunder except for that under a sheath opening to the needle slot in a proximal portion of the sheath body. The coupler includes a coupler housing and a valve module disposed in the coupler housing that seals the proximal portions of the needle shaft and the sheath body therein. The RICC insertion assembly also includes an access guidewire including a proximal end coupled to an extension arm of the coupler and a distal end disposed in the introducer needle. The RICC insertion assembly also includes a sheath-splitting means for splitting the sheath body away from the access guidewire subsequent to a percutaneous puncture with the introducer needle. The sheath-splitting means allows the access guidewire to escape from the sheath body and remain in a needle tract established by the percutaneous puncture. The splitting of the sheath body by the sheath-splitting means is initiated by a) withdrawing the sheath body by a pair of finger tabs extending from a sheath hub around the proximal portion of the sheath body, b) twisting the introducer needle or a needle hub around the proximal portion of the needle shaft, c) triggering one or more triggers disposed in the coupler housing, or d) pushing a catheter tip of the RICC into the sheath body.

In some embodiments, the splitting by a same or different sheath-splitting means is continued after initiation by pushing the coupler over the sheath body while holding the sheath body in place.

In some embodiments, the splitting by a same or different sheath-splitting means is continued after initiation by withdrawing the introducer needle from the coupler.

In some embodiments, the splitting by a same or different sheath-splitting means is continued after initiation by withdrawing the sheath body from the coupler.

In some embodiments, the splitting by a same or different sheath-splitting means is continued after initiation by pushing the catheter tip of the RICC into the sheath body.

In some embodiments, the splitting by a same or different sheath-splitting means is continued after initiation by pulling the access guidewire distally against a side of the sheath body.

In some embodiments, the sheath-splitting means is a blade, the access guidewire, the catheter tip, or a combination thereof.

In some embodiments, the blade is disposed in the valve module under a distal end of the sheath opening. The blade includes a distally facing blade edge configured to slide along the needle slot and cut the sheath body away from the access guidewire.

In some embodiments, the blade is spring-loaded on a compression spring. The compression spring is configured to relax when the introducer needle or the needle hub is twisted or the one-or-more triggers disposed in the coupler housing are triggered.

In some embodiments, the proximal and distal ends of the access guidewire enforce a loop in the access guidewire, over which loop the RICC is disposed in a ready-to-deploy state of the RICC insertion assembly.

In some embodiments, the RICC insertion assembly further includes a syringe fluidly coupled to the introducer needle in the ready-to-deploy state of the RICC insertion assembly. The sheath body seals the needle slot of the needle shaft thereunder outside of the valve module, the valve module seals the sheath opening of the sheath body therein, and the valve module seals around the access guidewire enabling the syringe to aspirate blood.

In some embodiments, the coupler housing includes a longitudinal coupler-housing slot configured to allow the access guidewire to escape from the coupler housing when the sheath body is split away from the access guidewire.

Also disclosed herein is a method for inserting a RICC into a blood-vessel lumen of a patient. The method includes, in some embodiments, a RICC insertion assembly-obtaining step, a needle tract-establishing step, an access guidewire-advancing step, a sheath body-splitting step, and a RICC-advancing step. The RICC insertion assembly-obtaining step includes obtaining a RICC insertion assembly. The RICC insertion assembly includes the RICC, an introducer needle including a sheath body over a needle shaft, and an access guidewire coupled together by a coupler. A proximal end of the access guidewire is coupled to an extension arm of the coupler and a distal end of the access guidewire is disposed in the introducer needle by way of a valve module of the coupler. The needle tract-establishing step establishes a needle tract from an area of skin to the blood-vessel lumen with the introducer needle. The access guidewire-advancing step includes advancing the distal end of the access guidewire from its initial location in the needle shaft just proximal of a needle tip of the needle shaft into the blood-vessel lumen. The sheath body-splitting step includes splitting the sheath body away from the access guidewire, thereby allowing the access guidewire to escape from the sheath body and remain in the needle tract. The sheath body-splitting step is initiated by a) withdrawing the sheath body by a pair of finger tabs extending from a sheath hub around the proximal portion of the sheath body, b) twisting the introducer needle or a needle hub around a proximal portion of the needle shaft, c) triggering one or more triggers disposed a coupler housing of the coupler, or d) pushing a catheter tip of the RICC into the sheath body. The RICC-advancing step includes advancing the catheter tip of the RICC over the access guidewire and into the blood-vessel lumen, thereby inserting the RICC into the blood-vessel lumen.

In some embodiments, the sheath body-splitting step is continued after initiation by pushing the coupler over the sheath body while holding the sheath body in place.

In some embodiments, the sheath body-splitting step is continued after initiation by withdrawing the introducer needle from the coupler.

In some embodiments, the sheath body-splitting step is continued after initiation by withdrawing the sheath body from the coupler.

In some embodiments, the sheath body-splitting step is continued after initiation by pushing the catheter tip of the RICC further into the sheath body with the advancing of the catheter tip over the access guidewire and into the blood-vessel lumen.

In some embodiments, the sheath body-splitting step is continued after initiation by pulling the access guidewire distally toward the patient and against a side of the sheath body.

In some embodiments, the splitting of the sheath body away from the access guidewire is accomplished with a blade.

In some embodiments, the blade is disposed in the valve module under a distal end of the sheath opening. The blade includes a distally facing blade edge configured to slide along the needle slot and cut the sheath body away from the access guidewire.

In some embodiments, the blade is spring-loaded on a compression spring. The compression spring is configured to relax when the introducer needle or the needle hub is twisted or the one-or-more triggers disposed in the coupler housing are triggered.

In some embodiments, the needle shaft includes a longitudinal needle slot extending from a proximal portion of the needle shaft through the needle tip. The needle slot allows the access guidewire to escape from the needle shaft with the continued splitting of the sheath body away from the access guidewire.

In some embodiments, the method further includes an access guidewire-withdrawing step. The access guidewire-withdrawing step includes withdrawing the access guidewire leaving the catheter tube in place in the blood-vessel lumen.

These and other features of the concepts provided herein will become more apparent to those of skill in the art in view of the accompanying drawings and following description, which describe particular embodiments of such concepts in greater detail.

### DRAWINGS

FIG. 1 illustrates a top view of a RICC insertion assembly in accordance with some embodiments.

FIG. 2 illustrates a bottom view of the RICC insertion assembly in accordance with some embodiments.

FIG. 3 illustrates a longitudinal cross section of a coupler and an introducer needle of the RICC insertion assembly in accordance with some embodiments.

FIG. 4 illustrates a longitudinal cross section of the coupler, the introducer needle, and an access guidewire of the RICC insertion assembly in accordance with some embodiments.

FIG. 5 illustrates a sheath of the introducer needle having a sheath hub with a pair of finger tabs configured for pulling the sheath away from the coupler to initiate splitting of the sheath in accordance with some embodiments.

FIG. 6 illustrates a button for a trigger disposed in a coupler housing of the coupler configured to initiate splitting of the sheath when the button is pressed in accordance with some embodiments.

FIG. 7 illustrates the introducer needle or a needle hub thereof configured to twist out from the coupler housing and initiate splitting of the sheath in accordance with some embodiments.

FIG. 8 illustrates a spring-loaded blade configured to initiate splitting of the sheath when triggered in accordance with some embodiments.

FIG. 9 illustrates a top view of the introducer needle in accordance with some embodiments.

FIG. 10 illustrates a sheath of the introducer needle in accordance with some embodiments.

FIG. 11 illustrates a needle shaft of the introducer needle in accordance with some embodiments.

FIG. 12 illustrates a RICC of the RICC insertion assembly in accordance with some embodiments.

FIG. 13 illustrates a detailed view of a distal portion of a catheter tube of the RICC in accordance with some embodiments.

FIG. 14 illustrates a transverse cross section of the distal portion of the catheter tube in accordance with some embodiments.

FIG. 15 illustrates another transverse cross section of the distal portion of the catheter tube in accordance with some embodiments.

FIG. 16 illustrates a longitudinal cross section of the distal portion of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are RICC insertion assemblies and methods of sheath removal that address the foregoing need. For example, a RICC insertion assembly can include a RICC, an introducer needle, a coupler coupling the RICC and the introducer needle together, and a sheath-splitting means for splitting a sheath body of the introducer needle away from an access guidewire. The sheath-splitting means allows the access guidewire to escape from the sheath body and remain in a needle tract established with the introduce needle. The splitting of the sheath body by the sheath-splitting means is initiated by withdrawing the sheath body by a pair of finger tabs extending from a sheath hub around the proximal portion of the sheath body, twisting the introducer needle or a needle hub around the proximal portion of the needle shaft, triggering one or more triggers disposed in the coupler housing, or pushing a catheter tip of the RICC into the sheath body.

The foregoing features as well as other features of the RICC insertion assemblies and methods thereof including sheath removal will become more apparent to those of skill in the art in view of the accompanying drawings and following description, which describe particular embodiments of the RICC insertion assemblies and methods thereof in greater detail. However, it should be understood the RICCs of the RICC insertion assemblies are but one type of catheter that can be incorporated into catheter insertion assemblies like those provided herein. Indeed, peripherally inserted central catheters (“PICCs”), dialysis catheters, or the like can also be incorporated into catheter insertion assemblies and methods.

### RICC Insertion Assemblies

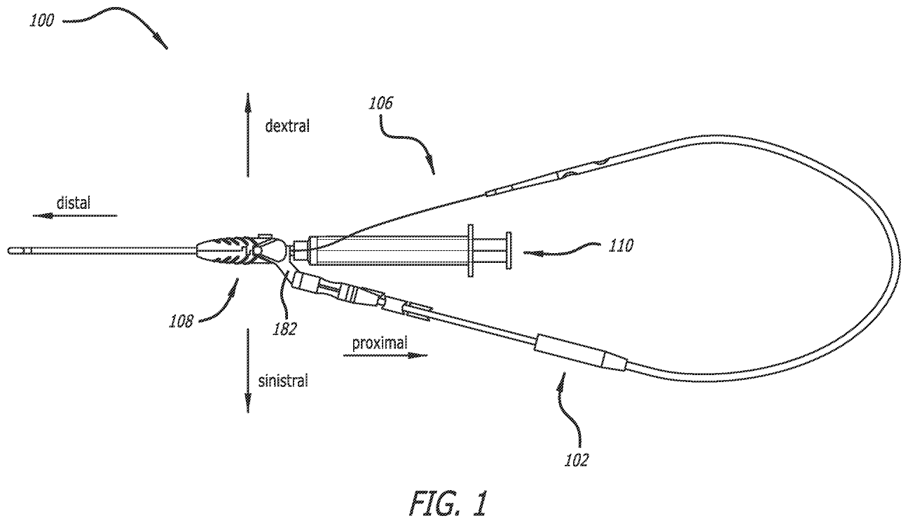

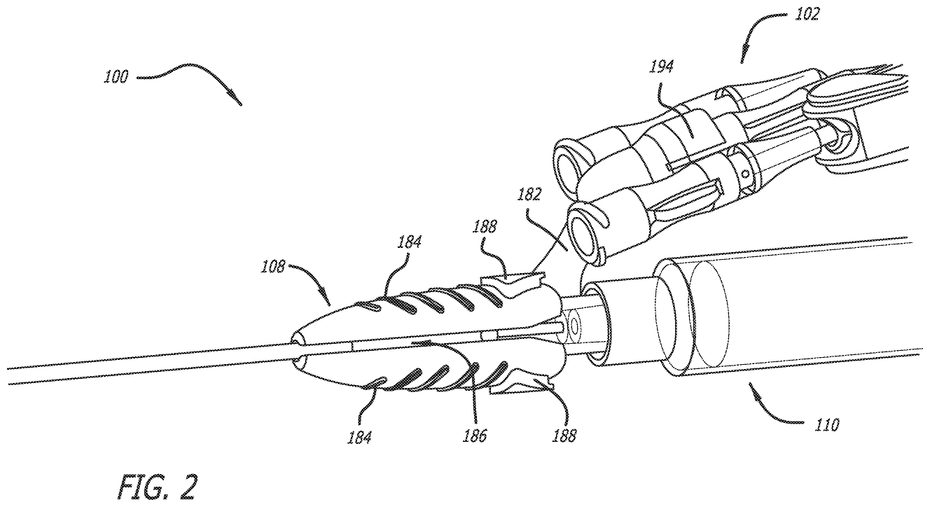

FIGS. 1 and 2 illustrate different views of a RICC insertion assembly 100 in accordance with some embodiments.

As shown, the RICC insertion assembly 100 includes a RICC 102, an introducer needle 104, an access guidewire 106, and a coupler 108 coupling the RICC 102, the introducer needle 104, and the access guidewire 106 together in a ready-to-deploy state of the RICC insertion assembly 100. Notably, the proximal end of the access guidewire 106 is coupled to the coupler 108 and the distal end of the access guidewire 106 is disposed in the needle lumen 158 of the introducer needle 104 as set forth below. This enforces a loop in the access guidewire 106, which loop the RICC 102 is disposed over in the ready-to-deploy state of the RICC insertion assembly 100 keeping the RICC insertion assembly 100 in a relatively compact form.

The RICC insertion assembly 100 can further include a syringe 110 fluidly coupled to the introducer needle 104 in the ready-to-deploy state of the RICC insertion assembly 100. As set forth below, the sheath 141 or the sheath body 142 thereof seals the needle slot 148 of the needle shaft 140. In particular, the sheath body 142 seals the needle slot 148 outside of the valve module 180. The valve module 180, in turn, seals over the sheath opening 162 of the sheath body 142 that opens to the needle slot 148. The valve module 180 also seals around the access guidewire 106. Such seals enable the syringe 110 to aspirate blood in accordance with the blood-aspirating step of the method set forth below.

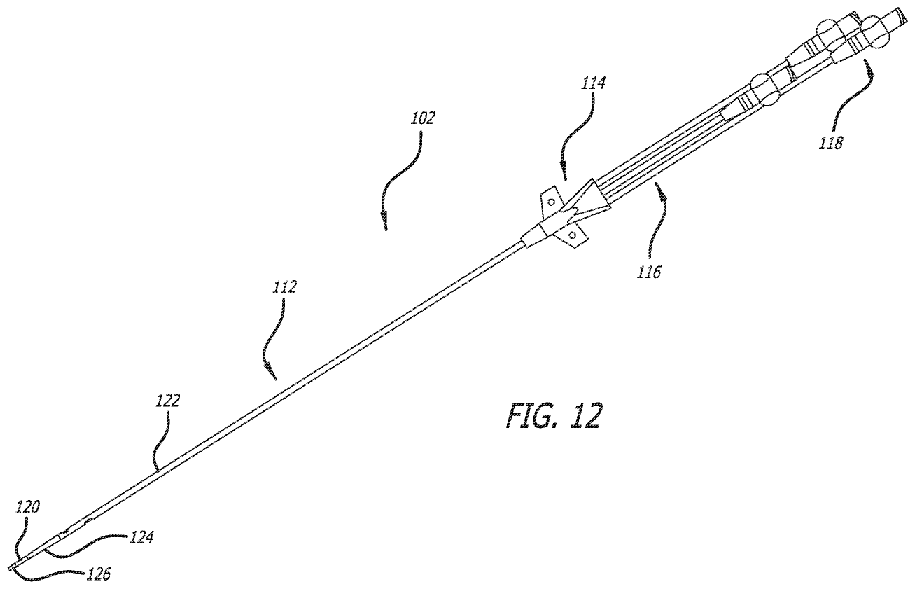

FIG. 12 illustrates the RICC 102 of the RICC insertion assembly 100 in accordance with some embodiments.

As shown, the RICC 102 includes a catheter tube 112, a catheter hub 114, one or more extension legs 116, and one or more extension-leg connectors 118.

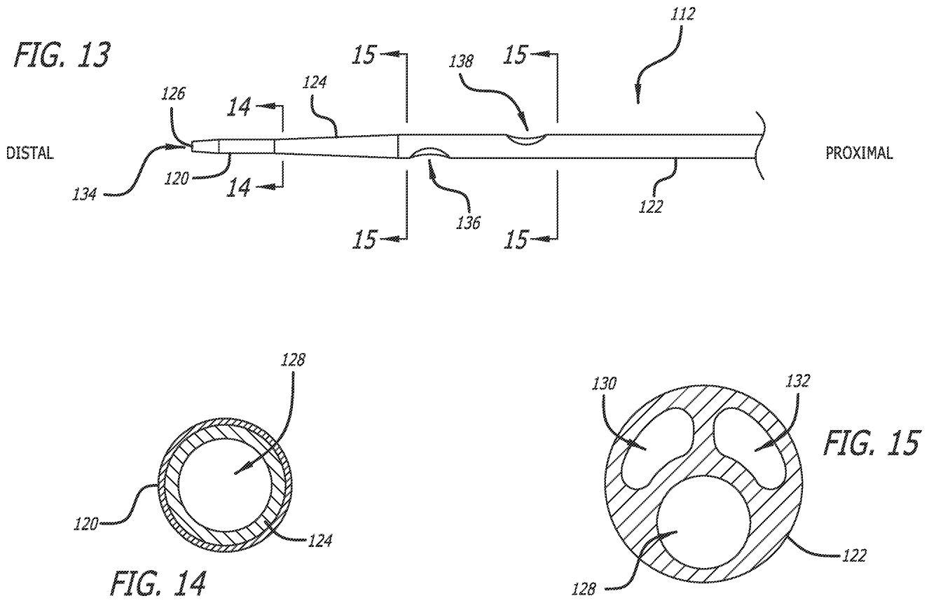

FIGS. 13-16 illustrate various views of the catheter tube 112 of the RICC 102 in accordance with some embodiments.

The catheter tube 112 includes a first section 120 in a distal portion of the catheter tube 112, a second section 122 in the distal portion of the catheter tube 112 proximal of the first section 120, and a tapered junction 124 between the first and second sections 120 and 122 of the catheter tube 112.

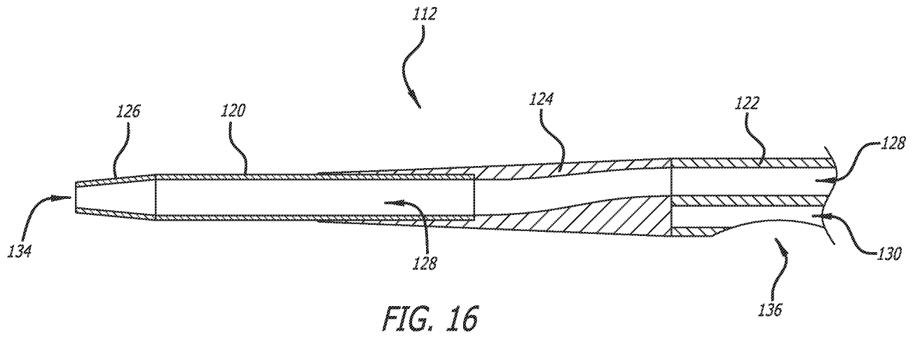

The first section 120 of the catheter tube 112 includes a catheter tip 126 having a relatively short taper from an outer diameter of a distal portion of the first section 120 distal of the junction 124 to an outer diameter of a distal end of the first section 120. The taper of the catheter tip 126 is configured for immediate dilation of tissue about a needle tract established with the introducer needle 104 up to the outer diameter of the distal portion of the first section 120 of the catheter tube 112. As best shown in FIG. 16, the first section 120 of the catheter tube 112 also includes a proximal portion disposed in a bore of a distal portion of the junction 124 and fixedly coupled thereto such as by a solvent bond, an adhesive bond, or a heat weld.

The second section 122 of the catheter tube 112 includes a consistent outer diameter over its length from a distal end of the second section 122 to a proximal end of the second section 122. The consistent diameter of the second section 122 of the catheter tube 112 is configured for smooth insertion into the needle tract and targeted vasculature subsequent to any dilation by the first section 120 of the catheter tube 112 and the junction 124. The distal end of the second section 122 of the catheter tube 112 has a flat face flush with the flat-faced proximal end of the junction 124 and fixedly coupled thereto such as by a solvent bond, an adhesive bond, or a heat weld.

The junction 124 includes a taper over its length from a proximal end of the junction 124 to a distal end of the junction 124. The taper of the junction 124 is configured for immediate dilation of the tissue about the needle tract from the outer diameter of the proximal portion of the first section 120 of the catheter tube 112 to the outer diameter of the second section 122 of the catheter tube 112. An abluminal surface of the junction 124 smoothly transitions from an abluminal surface of the first section 120 of the catheter tube 112 to an abluminal surface of the second section 122 of the catheter tube 112 without edges that catch on skin when the catheter tube 112 is inserted into the needle tract. In addition to the edges being minimal to negligible, the edges can include solvent-interdiffused polymeric material of the polymeric materials from which the catheter tube 112 is formed, which smoothens the transitions from the first section 120 of the catheter tube 112 to the junction 124 and from the junction 124 to the second section 122 of the catheter tube 112. Notably, the junction 124 has a length approximately commensurate with a length of an exposed portion of the first section 120 of the catheter tube 112 or between lengths of exposed portions of the first and second sections 120 and 122 of the catheter tube 112. As such, the length of the exposed portion of the first section 120 of the catheter tube 112 is less than the length of the junction 124 up to approximately commensurate with the length of the junction 124.

The first section 120 of the catheter tube 112 is formed of a first polymeric material (e.g., a polytetrafluoroethylene, a polypropylene, or a polyurethane) having a first durometer. The second section 122 of the catheter tube 112 is formed of a second polymeric material (e.g., a polyvinyl chloride, a polyethylene, another polyurethane, or a silicone) having a second durometer less than the first durometer. For example, the first section 120 of the catheter tube 112 can be formed of a first polyurethane having the first durometer while the second section 122 of the catheter tube 112 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 112 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 124 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 120 of the catheter tube 112, the second section 122 of the catheter tube 112, and the junction 124 between the first and second sections 120 and 122 of the catheter tube 112 set forth above, the catheter tube 112 possesses a column strength sufficient to prevent buckling of the catheter tube 112 when inserted into a needle tract established by with the introducer needle 104, optionally, over the access guidewire 106. The column strength of the catheter tube 112 is also sufficient to prevent buckling of the catheter tube 112 when advanced through a vasculature of a patient, optionally, over a maneuver guidewire, without dilation of tissue about the needle tract or any blood vessels of the vasculature beforehand with a separate dilator. The column strength of the catheter tube 112 is also sufficient to prevent buckling of the catheter tube 112 when the catheter tip 126 is pushed into the sheath body 142 to split the sheath body 142 away from the access guidewire 106 in accordance with the sheath-splitting step of the method set forth below.

The catheter tube 112 includes one or more catheter-tube lumens extending through the catheter tube 112; however, only one catheter-tube lumen typically extends from a proximal end of the catheter tube 112 to a distal end of the catheter tube 112 in a multiluminal RICC (e.g., a diluminal RICC, a triluminal RICC, a tetraluminal RICC, a pentaluminal RICC, a hexaluminal RICC, etc.). (See FIGS. 13-16.) Indeed, the first section 120 of the catheter tube 112 typically includes a single lumen therethrough as shown in FIGS. 14, 15, and 16.

The catheter hub 114 is coupled to a proximal portion of the catheter tube 112. The catheter hub 114 includes one or more catheter-hub lumens corresponding in number to the one-or-more catheter-tube lumens. The one-or-more catheter-hub lumens extends through an entirety of the catheter hub 114 from a proximal end of the catheter hub 114 to a distal end of the catheter hub 114.

Each extension leg of the one-or-more extension legs 116 is coupled to the catheter hub 114 by a distal portion thereof. The one-or-more extension legs 116 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 118 is over a proximal portion of an extension leg of the one-or-more extension legs 116. For example, each extension-leg connector of the one-or-more extension-leg connectors 118 can be a Luer connector over a proximal portion of an extension leg of the one-or-more extension legs 116. Through such an extension-leg connector, a corresponding extension leg and the extension-leg lumen thereof can be connected to another medical device and a lumen thereof. However, in the ready-to-deploy state of the RICC insertion assembly 100 at least one extension-leg connector (e.g., the extension-leg connector including part of the primary lumen 128 of the RICC 102) is connected to the extension-arm connector 194 of the extension arm 182 of the coupler 108 to enforce the loop in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 128, a secondary lumen 130, and a tertiary lumen 132 formed of fluidly connected portions of three catheter-tube lumens, three catheter-hub lumens, and three extension-leg lumens. The primary lumen 128 has a primary-lumen aperture 134 in the distal end of the first section 120 of the catheter tube 112, which corresponds to the distal end of the catheter tube 112 and a distal end of the RICC 102. The secondary lumen 130 has a secondary-lumen aperture 136 in a side of the distal portion of the catheter tube 112. The tertiary lumen 132 has a tertiary-lumen aperture 138 in the side of the distal portion of the catheter tube 112 proximal of the secondary-lumen aperture 136.

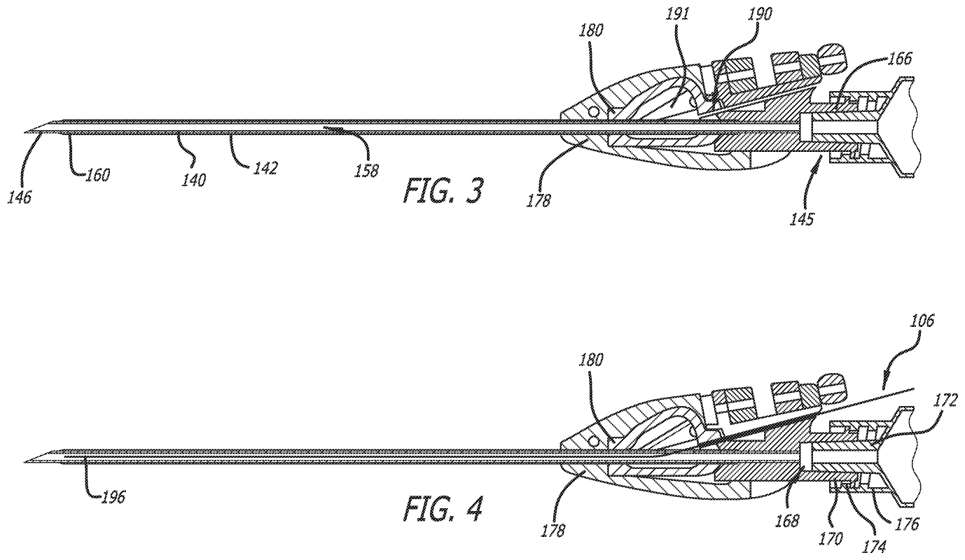

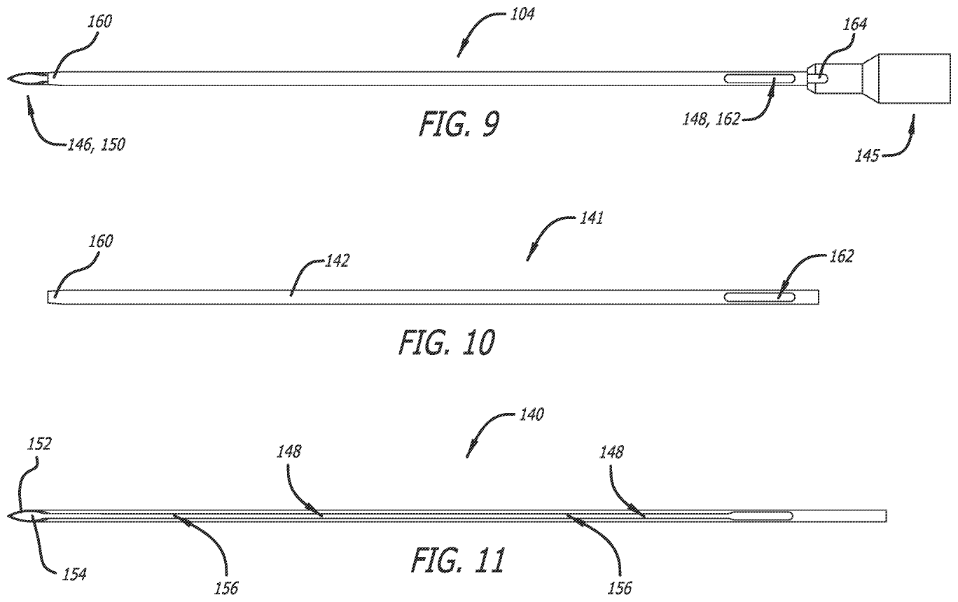

FIGS. 3, 4, 5, and 9-11 illustrate various views of the introducer needle 104 of the RICC insertion assembly 100 in accordance with some embodiments.

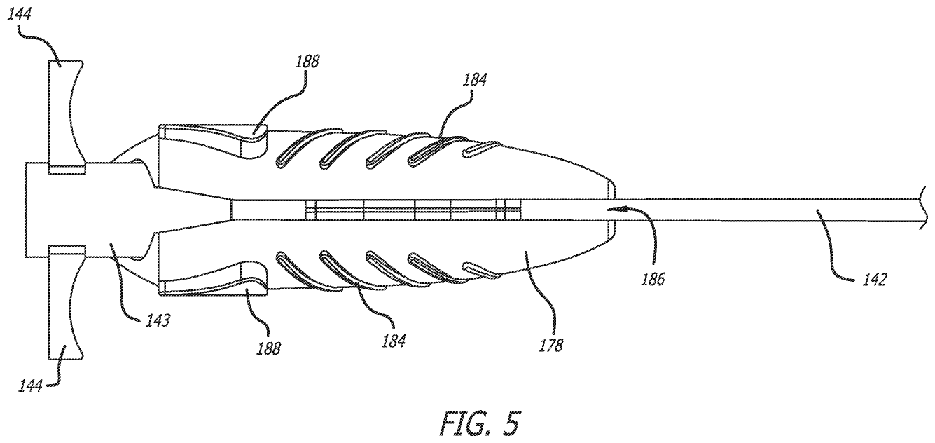

As shown in FIGS. 3, 4, and 9-11, the introducer needle 104 includes a needle shaft 140, a sheath 141 or a sheath body 142 thereof over the needle shaft 140, and a needle hub 145 over both a proximal portion of the needle shaft 140 and a proximal portion of the sheath body 142. However, as shown in FIG. 5, the sheath 141 can alternatively include a sheath hub 143 around the proximal portion of the sheath body 142, wherein the sheath hub 143 is configured to nest with the needle hub 145 with the sheath hub 143 over the proximal portion of the sheath body 142 and the needle hub 145 over the proximal portion of the needle shaft 140. In at least the ready-to-deploy state of the RICC insertion assembly 100, the needle shaft 140 and the sheath body 142 extend from the needle hub 145 and, optionally, from the sheath hub 143, respectively, through the valve module 180, and out a distal end of the coupler housing 178.

The needle shaft 140 includes a needle tip 146 in a distal portion of the needle shaft 140 and a longitudinal needle slot 148 extending from the proximal portion of the needle shaft 140 through the needle tip 146.

The needle tip 146 includes a bevel 150 having a tip bevel 152 and a primary bevel 154 proximal of the tip bevel 152. A tip-bevel angle of the tip bevel 152 is greater than a primary-bevel angle of the primary bevel 154 such that the bevel 150 provides a smooth transition over the needle tip 146. Such a needle tip is thusly configured for establishing a needle tract from an area of skin into a blood-vessel lumen of a patient in accordance with the needle tract-establishing step of the method set forth below.

The needle slot 148 extends from the proximal portion of the needle shaft 140 through the needle tip 146, thereby forming a needle channel 156 along a majority of a length of the needle shaft 140 as opposed to a needle lumen therethrough. The needle slot 148 has a width sized in accordance with an outer diameter of the access guidewire 106, which allows the access guidewire 106 to pass from the proximal portion of the needle shaft 140 through the needle tip 146 when the introducer needle 104 or the needle shaft 140 thereof is proximally withdrawn from the coupler 108.

While the needle shaft 140 includes the foregoing needle slot 148, it should be understood the introducer needle 104 includes a needle lumen 158; however, the needle lumen 158 results from the combination of the needle shaft 140 and the sheath body 142 over the needle shaft 140. Indeed, the sheath body 142 over the needle shaft 140 seals the needle slot 148 thereunder forming the needle lumen 158 of the introducer needle 104 and enabling the syringe 110 to aspirate blood in accordance with the blood-aspirating step of the method set forth below.

The sheath 141 includes a sheath tip 160 in a distal portion of the sheath body 142 as well as a sheath opening 162 in a side of the proximal portion of the sheath body 142.

The sheath tip 160 includes a relatively short taper from an outer diameter of the distal portion of the sheath body 142 to an outer diameter of a distal end of the sheath 141, the latter of which is commensurate with an outer diameter of the distal portion of the needle shaft 140. The taper has a taper angle less than the primary-bevel angle of the primary bevel 154 of the needle tip 146, which, in turn, is less than the tip-bevel angle of the tip bevel 152 of the needle tip 146. The sheath tip 160 including such a taper is configured to provide a smooth transition from the needle tip 146 to the sheath body 142 for the needle tract-establishing step of the method set forth below.

The sheath opening 162 opens to the needle slot 148 of the needle shaft 140 allowing the access guidewire 106 to pass through the sheath opening 162 and into the needle slot 148 in the ready-to-deploy state of the RICC insertion assembly 100. Thus, the sheath opening 162 has a width approximately commensurate with a width of the needle slot 148, which, in turn, is sized in accordance with the diameter of the access guidewire 106. The sheath opening 162 also has a length sufficient to allow the access guidewire 106 to pass through the sheath opening 162 and into the needle slot 148 while also accommodating the stationary blade 191 of the valve module 180, when present, under a distal end of the sheath opening 162. Notably, the sheath body 142 over the needle shaft 140 seals the needle slot 148 thereunder except for that under the sheath opening 162. However, the valve module 180 seals over the needle slot 148 exposed by the sheath opening 162 by sealing the proximal portions of the needle shaft 140 and the sheath body 142 therein, thereby enabling the syringe 110 to aspirate blood in accordance with the blood-aspirating step of the method set forth below.

The sheath 141, or the sheath body 142 thereof, is formed of a polymeric material configured to facilitate a smooth, consistent insertion of the introducer needle 104 from an area of skin to a blood-vessel lumen of a patient in accordance with the needle tract-establishing step of the method set forth below. In addition, the polymeric material has mechanical properties at a thickness of the sheath body 142 sufficient to withstand collapse of the sheath body 142 into the needle slot 148 of the needle shaft 140 when the blood-aspirating step of the method set forth below is performed, notably, while also facilitating the splitting of the sheath body 142 away from the access guidewire 106 in accordance with the sheath-splitting step of the method set forth below whether by way of cutting the sheath body 142 with the stationary or moveable blade 191 or 192, pushing the catheter tip 126 of the RICC 102 into the sheath body 142, or pulling the access guidewire 106 distally against the side of the sheath body 142. Such a polymeric material can include, but is not limited to, polyethylene, polypropylene, or polytetrafluoroethylene.

The needle hub 145 includes an access-guidewire channel 164 in a distal portion of the needle hub 145 and a needle-hub connector 166 in a proximal portion of the needle hub 145.

The access-guidewire channel 164 of the needle hub 145 is configured to allow the access guidewire 106 to pass over the needle hub 145 and direct the access guidewire 106 into the access-guidewire conduit 190 of the valve module 180. The access-guidewire channel 164 is open such that the access guidewire 106 lies in the access-guidewire channel 164 in at least the ready-to-deploy state of the RICC insertion assembly 100. Advantageously, the open access-guidewire channel 164 allows the access guidewire 106 to remain in place when the introducer needle 104 of the needle shaft 140 is withdrawn from the RICC insertion assembly 100 or the coupler 108 thereof.

The needle-hub connector 166 includes a needle-hub bore 168 and an optional needle-hub flange 170 about the needle-hub connector 166.

The needle-hub bore 168 of the needle-hub connector 166 is configured to accept a syringe tip 172 of the syringe 110 therein for fluidly connecting the introducer needle 104 to the syringe 110. Indeed, the needle-hub bore 168 can have a Luer taper (e.g., a 6% taper) configured to accept the syringe tip 172 therein, which syringe tip 172 can be complementarily configured with a Luer taper.

The needle-hub flange 170 of the needle-hub connector 166 is configured to screw together with internal threads 174 of a threaded collar 176 around the syringe tip 172 of the syringe 110. While the threaded collar 176 of the syringe 110 is optional, the needle-hub flange 170 advantageously provides a so-called Luer lock-style connection with the internal threads 174 of the threaded collar 176 when both are present. This provides added security against inadvertent disconnection of the introducer needle 104 and the syringe 110 over that provided by an otherwise Luer slip-style connection.

When present, the sheath hub 143 includes a pair of finger tabs 144 extending from the sheath hub 143 configured for at least withdrawing the sheath 141 from the coupler 108 by the finger tabs 144. Advantageously, the splitting of the sheath body 142 in accordance with the sheath-body-splitting step of the method set forth below can be initiated by withdrawing the sheath 141 from the coupler 108 by the finger tabs 144. Indeed, the withdrawing of the sheath 141 from the coupler 108 by the finger tabs 144 can initiate cutting the sheath 141 with the stationary blade 191 set forth below when the stationary blade 191 is also present. In addition, the cutting of the sheath body 142 by the stationary blade 191 or even the moveable blade 192 set forth below can be continued after initiation by continued withdrawing of the sheath 141 from the coupler 108 by the finger tabs 144. Alternatively, the sheath hub 143 is splittable and the splitting of the sheath body 142 is initiated by pulling the finger tabs 144 away from each other to split the sheath hub 143 after the sheath hub 143 is withdrawn from the coupler 108 and free thereof. In addition, the splitting of the sheath body 142 can be continued after initiation by continued pulling of the finger tabs 144 away from each other. Notably, with a 90° rotation of the sheath 141 from that shown in FIG. 5, a longitudinal half of the sheath body 142 can be split from another longitudinal half of the sheath body 142 and out of the coupler 108 by way of the coupler-housing slot 186, thereby allowing the access guidewire 106 to escape from the coupler 108 after the sheath 141 is completely split.

FIGS. 3-8 illustrate various view of the coupler 108 of the RICC insertion assembly 100 in accordance with some embodiments.

As shown, the coupler 108 includes a coupler housing 178 and a valve module 180 disposed in the coupler housing 178. In addition, the coupler 108 can include an extension arm 182 coupled to the coupler housing 178.

The coupler housing 178 includes two molded halves coupled together to form an ovoid body configured to be comfortably held underhand (e.g., cradled) or overhand in either a left hand for a left-handed venipuncture or a right hand for a right-handed venipuncture with the RICC insertion assembly 100. (It should be understood, however, the coupler housing 178 can alternatively be two or more pieces other than the two molded halves.) To further facilitate such venipunctures, an outside of each half of the two molded halves can be textured as shown with grip-enhancing arcuate ridges 184 or the like. An inside of each half of the two molded halves includes depressions that form a valve-module compartment and an introducer-needle receptacle when the two molded halves are coupled together as shown. (See FIGS. 3 and 4, which include the valve module 180 disposed in a depression of a molded half of the two molded halves that form the valve-module compartment. FIGS. 3 and 4 also include the needle hub 145 of the introducer needle 104 disposed in a depression of the molded half of the two molded halves that form the introducer-needle receptacle. However, FIG. 5 includes the sheath hub 143 of the introducer needle 104 disposed in the introducer-needle receptacle.) In addition, each half of the two molded halves can include one or more through holes or portions thereof for a corresponding one or more triggers such as one or more buttons, switches, sliders etc. (See, for example, FIGS. 2, 5, and 6 for the pair of buttons 188 extending through the corresponding pair of through holes therefor.) Notably, the coupler housing 178 includes a longitudinal coupler-housing slot 186 formed between the two molded halves. The coupler-housing slot 186 is configured to allow the access guidewire 106 to escape from the coupler housing 178 when the introducer needle 104 or the needle shaft 140 thereof is proximally withdrawn from the coupler 108.

The valve-module compartment is configured to hold the valve module 180 therein. Indeed, the valve-module compartment includes the valve module 180 disposed therein in the ready-to-deploy state of the RICC insertion assembly 100. Notably, the valve-module compartment is further configured with sufficient space to allow the separable pieces of the valve module 180 set forth below to separate for the escape of the access guidewire 106 when the introducer needle 104 or the needle shaft 140 thereof is proximally withdrawn from the coupler 108.

The introducer-needle receptacle is configured to hold the needle hub 145 or the sheath hub 143 of the introducer needle 104 therein. As shown in FIGS. 3 and 4, for example, the introducer-needle receptacle includes the needle hub 145 inserted therein in the ready-to-deploy state of the RICC insertion assembly 100. However, as shown in FIG. 5, the introducer-needle receptacle includes the sheath hub 143 inserted therein in the ready-to-deploy state of the RICC insertion assembly 100. Notably, an introducer-needle lock configured to lock the needle hub 145 or the sheath hub 143 in the introducer-needle receptacle can be positioned about the introducer-needle receptacle. When the introducer-needle lock is present, a pair of buttons 188 can be configured as spring-loaded lock buttons of the introducer-needle lock. Such a pair of buttons can be distributed between opposing sides of the coupler 108, particularly in a pair of through holes therefor in the two molded halves of the coupler housing 178, such that each button of the pair of buttons 188 extends through the coupler housing 178 on its respective side of the coupler 108. In addition, such a pair of buttons can be configured to unlock the needle hub 145 or the sheath hub 143 when are triggered by pressing the pair of buttons 188 into the coupler 108 for subsequent withdrawal of the introducer needle 104 from the coupler 108.

The valve module 180 includes and a number of separable pieces, which, when combined as shown in at least FIGS. 3 and 4, form an access-guidewire conduit 190. In addition, the valve module 180 can include a stationary blade 191 disposed therein.

The separable pieces of the valve module 180 are disposed around the needle shaft 140 and the sheath body 142 in the ready-to-deploy state of the RICC insertion assembly 100. For example, the separable pieces of the valve module 180 can be separable halves of the valve module 180 disposed around the needle shaft 140 and the sheath body 142. The separable pieces of the valve module 180 are configured to separate and allow the access guidewire 106 to escape from the valve module 180 when the introducer needle 104 or the needle shaft 140 thereof is proximally withdrawn from the coupler 108.

The access-guidewire conduit 190 is configured to direct the access guidewire 106 from the access-guidewire channel 164 of the needle hub 145 into both the sheath opening 162 of the sheath 141 or the sheath body 142 thereof and the needle slot 148 of the needle shaft 140 thereunder. Indeed, the access-guidewire conduit 190 includes the access guidewire 106 disposed therein in the ready-to-deploy state of the RICC insertion assembly 100. Notably, the valve module 180 seals around the access guidewire 106 in the access-guidewire conduit 190 such that the syringe 110 is able to aspirate blood in accordance with the blood-aspirating step of the method set forth below.

When present, the stationary blade 191 extends from an attachment point in the valve module 180 into the needle slot 148 of the needle shaft 140 such that the stationary blade 191 is disposed in the needle slot 148 under the distal end of the sheath opening 162 of the sheath body 142. The stationary blade 191 is configured to slide along the needle slot 148 as either the introducer needle 104 or the needle shaft 140 thereof is withdrawn in a proximal direction from the coupler 108. The stationary blade 191 includes a distally facing blade edge configured to cut the sheath body 142 away from the access guidewire 106 as either the introducer needle 104 or the sheath body 142 thereof is withdrawn in the proximal direction from the coupler 108. Indeed, when the introducer needle 104 is withdrawn in its entirety from the coupler 108 in the sheath-splitting step of the method set forth below, the stationary blade 191 is configured to slide along the needle slot 148 and the blade edge is configured to cut the sheath body 142 away from the access guidewire 106 as the introducer needle 104 is withdrawn in the proximal direction from the coupler 108. However, when the introducer needle 104 is withdrawn piecemeal from the coupler 108, the stationary blade 191 is configured to slide along the needle slot 148 as the needle shaft 140 is withdrawn in the proximal direction from the coupler 108 and the blade edge is configured to cut the sheath body 142 away from the access guidewire 106 as the sheath body 142 is withdrawn in the proximal direction from the coupler 108. Cutting the sheath body 142 away from the access guidewire 106 allows the access guidewire 106 to escape from the needle shaft 140 by way of the needle slot 148. Notably, the cutting of the sheath body 142 by the stationary blade 191 can be continued after initiation of the cutting by continued withdrawing of the sheath 141 from the coupler 108 in the proximal direction.

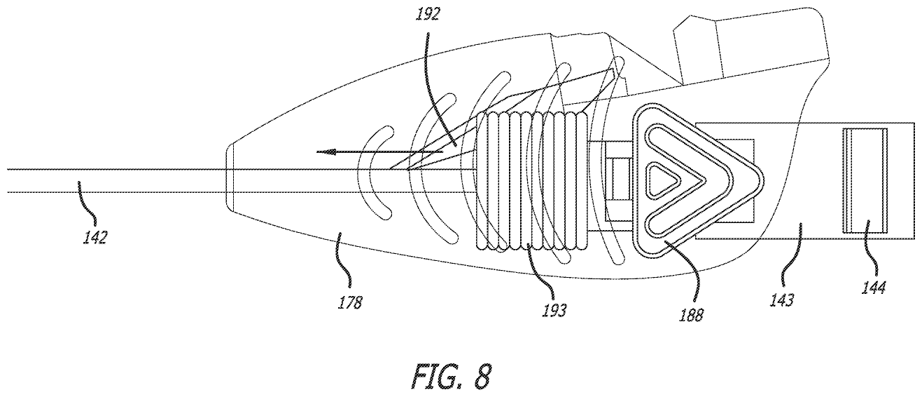

As an alternative to the stationary blade 191, a moveable blade 192 can be spring-loaded on a compression spring 193 as shown in FIG. 8. Such a movable blade can be located inside or outside of the valve module 180. Like the stationary blade 191, the moveable blade 192 is disposed in the needle slot 148 under the distal end of the sheath opening 162 of the sheath body 142 when located inside of the valve module 180. Further like the stationary blade 191, the moveable blade 192 is configured to slide along the needle slot 148 as either the introducer needle 104 or the needle shaft 140 thereof is withdrawn in the proximal direction from the coupler 108. Also, the moveable blade 192 includes a distally facing blade edge configured to cut the sheath body 142 away from the access guidewire 106 as either the introducer needle 104 or the sheath body 142 thereof is withdrawn in the proximal direction from the coupler 108. However, the cutting of the sheath body 142 away from the access guidewire 106 is initiated by triggering the one-or-more triggers, which, in turn, distally thrusts the moveable blade 192 a short distance along the needle slot 148 as the compression spring 193 relaxes. Notably, the cutting of the sheath body 142 by the moveable blade 192 can be continued after initiation of the cutting by continued withdrawing of the sheath 141 from the coupler 108 in the proximal direction.

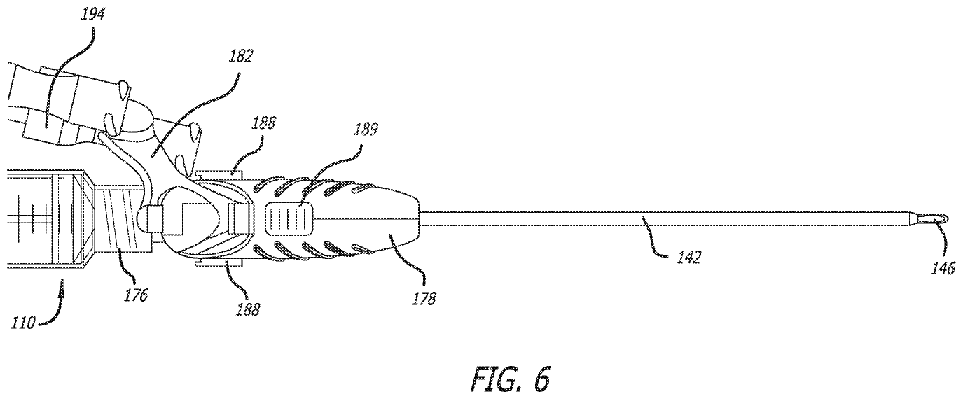

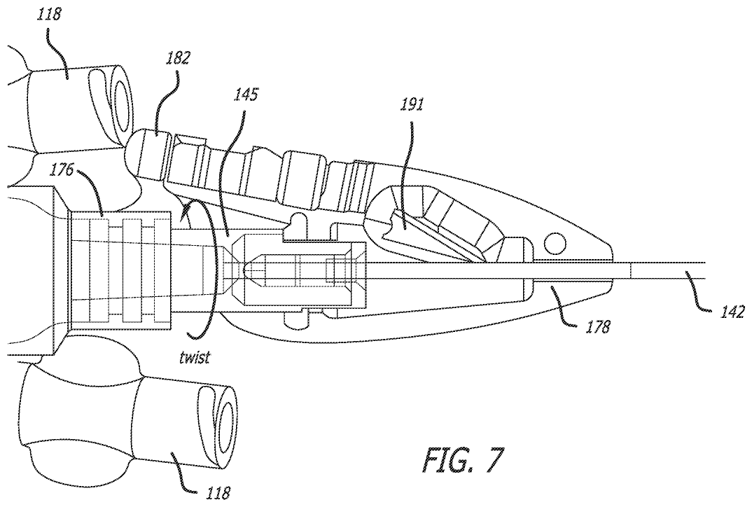

The one-or-more triggers for the spring-loaded moveable blade 192 can include the pair of buttons 188, which can be configured to unlock the needle hub 145 or the sheath hub 143 as well as release the compression spring 193 when triggered by pressing the pair of buttons 188 into the coupler 108. With or without the pair of buttons 188, the one-or-more triggers can include another trigger such as a slider 189 as shown in FIG. 6. With the pair of buttons 188, the other trigger such as the slider 189 can be configured to release the compression spring 193 when triggered by sliding the slider 189 along the coupler housing 178 either before or after the pair of buttons 188 are pressed into the coupler 108 to unlock the needle hub 145 or the sheath hub 143. Without the pair of buttons 188, the other trigger such as the slider 189 can be configured to unlock the needle hub 145 or the sheath hub 143 as well as release the compression spring 193 when triggered by sliding the slider 189 along the coupler housing 178. However, it should be understood that the one-or-more triggers are not limited to the one-or-more buttons, switches, or sliders. Indeed, in some embodiments, simply twisting the introducer needle 104 or a component thereof such as the needle hub 145 or the sheath hub 143 within the introducer-needle receptacle can unlock the introducer needle 104 or the needle hub 145 or the sheath hub 143 thereof, release the compression spring 193 to initiate the cutting of the sheath body 142 away from the access guidewire 106, or both unlock the introducer needle 104 or the needle hub 145 or the sheath hub 143 thereof and release the compression spring 193 to initiate the cutting of the sheath body 142 away from the access guidewire 106. (See, for example, FIG. 7 for at least twisting the introducer needle 104 or a component thereof such as the needle hub 145 or the sheath hub 143 to unlock the needle hub 145 or the sheath hub 143.

The extension arm 182 includes an extension-arm connector 194 connected to an extension-leg connector of the one-or-more extension-leg connectors 118 in the ready-to-deploy state of the RICC insertion assembly 100. While not shown, the extension-arm connector 194 includes an access-guidewire attachment point within the extension-arm connector 194 to which the proximal end of the access guidewire 106 is attached in the ready-to-deploy state of the RICC insertion assembly 100. In combination with the distal end of the access guidewire 106 being disposed in the needle lumen 158 of the introducer needle 104, the loop in the access guidewire 106 set forth above is enforced. Advantageously, the extension arm 182 is configured to flip the loop—or at least the one-or-more extension legs 116 of the RICC 102 thereof—between a sinistral side of the RICC insertion assembly 100 and a dextral side of the RICC insertion assembly 100 to accommodate both left-handed and right-handed venipunctures with the RICC insertion assembly 100. Indeed, the extension arm 182 is configured to flip the loop from the sinistral side of the RICC insertion assembly 100 as shown in FIG. 1 to the dextral side of the RICC insertion assembly 100 to accommodate a left-handed venipuncture with the RICC insertion assembly 100. Likewise, the extension arm 182 is configured to flip the loop from the dextral side of the RICC insertion assembly 100 to the sinistral side of the RICC insertion assembly 100 to accommodate a right-handed venipuncture with the RICC insertion assembly 100.

FIGS. 1 and 4 illustrate different views of the access guidewire 106 of the RICC insertion assembly 100 in accordance with some embodiments.

The access guidewire 106 includes a proximal portion including a proximal end and a distal portion including a distal end. In the ready-to-deploy state of the RICC insertion assembly 100, the proximal end of the access guidewire 106 is coupled to the extension arm 182, particularly the access-guidewire attachment point within the extension-arm connector 194 of the extension arm 182. In addition, the proximal portion of the access guidewire 106 extends along the primary lumen 128 of the RICC 102. The distal portion of the access guidewire 106 also extends along the primary lumen 128 of the RICC 102, but the distal portion of the access guidewire 106 further extends out the distal end of the RICC 102, into the valve module 180 over the needle hub 145 by way of the access-guidewire channel 164, into the needle shaft 140 through both the sheath opening 162 of the sheath body 142 and the needle slot 148 of the needle shaft 140, and along the needle lumen 158 of the introducer needle 104 in the ready-to-deploy state of the RICC insertion assembly 100. As shown in FIG. 4, the distal end of the access guidewire 106 is disposed in the needle lumen 158 just proximal of the needle tip 146 in the ready-to-deploy state of the RICC insertion assembly 100. Again, the proximal and distal ends of the access guidewire 106 enforce the loop in the access guidewire 106 in the ready-to-deploy state of the RICC insertion assembly 100, which loop the RICC 102 is disposed over, thereby keeping the RICC insertion assembly 100 in a relatively compact form.

The access guidewire 106 can include a guidewire tip 196 in the distal portion of the access guidewire 106, which adopts a T shape configured to prevent puncturing a back wall of a blood vessel. Such a guidewire tip assumes a straightened state in the ready-to-deploy state of the RICC insertion assembly 100 and a curved state when the guidewire tip 196 is advanced beyond the needle tip 146 (e.g., advanced into a blood-vessel lumen) in a deployed state of the RICC insertion assembly 100.

The access guidewire 106 can further include a bare-wire portion and a wound-wire portion proximal of the bare-wire portion. While not shown, the bare-wire portion, when present, distally extends through the access-guidewire conduit 190 of the valve module 180 in at least the ready-to-deploy state of the RICC insertion assembly 100 such that the valve module 180 forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings. Regardless of whether the access guidewire 106 includes any one of the foregoing bare-wire, round-wound, flat-wound, or ground-wound portions of the access guidewire 106, the access guidewire 106 can be configured with a tensile strength sufficient to pull the access guidewire 106 through the sheath body 142. Indeed, in some embodiments, the splitting or cutting of the sheath body 142 away from the access guidewire 106 is initiated by pulling the access guidewire 106 distally against a side of the sheath body 142, continued by pulling the access guidewire 106 distally against the side of the sheath body, or both initiated and continued by pulling the access guidewire 106 distally against the side of the sheath body 142.

As set forth above, the RICC insertion assembly 100 includes a number of sheath-splitting means such as the stationary or moveable blade 191 or 192, the access guidewire 106, or the catheter tip 126 and associated manners for splitting the sheath body 142 away from the access guidewire 106 subsequent to establishing a needle tract to a blood-vessel lumen by way of a percutaneous puncture with the introducer needle 104. The sheath-splitting means and the manner in which it splits the sheath body 142 away from the access guidewire 106 is notable for it allows the access guidewire 106 to escape from the sheath body 142 and remain in the needle tract for subsequent procedural steps in placing the RICC 102 in a patient. As set forth above, the splitting of the sheath body 142 by can be initiated by withdrawing the introducer needle 104 or the sheath body 142 by the pair of finger tabs 144 extending from the sheath hub 143 around the proximal portion of the sheath body 142 to initiate cutting of the sheath body 142 with the stationary blade 191; twisting the introducer needle 104, the needle hub 145, or the sheath hub 143 around the proximal portion thereof to initiate cutting of the sheath body 142 with the moveable blade 192; triggering the one-or-more triggers disposed in the coupler housing 178 to initiate cutting of the sheath body 142 with the moveable blade 192; pushing the catheter tip 126 of the RICC 102 into the sheath body 142 to initiate splitting of the sheath body 142 with the catheter tip 126; or pulling the access guidewire 106 distally against the side of the sheath body 142 to initiate splitting of the sheath body 142 with the access guidewire 106. After the splitting of the sheath body 142 is initiated, the splitting of the sheath body 142 can be continued by a same or different sheath-splitting means than that used to initiate the splitting of the sheath body 142.

In an example, the splitting of the sheath body 142 by can be continued by the same sheath-splitting means such as by continuing to withdraw the introducer needle 104 or the sheath body 142 by the pair of finger tabs 144 and cut the sheath body 142 with the stationary or moveable blade 191 or 192; push the catheter tip 126 of the RICC 102 into the sheath body 142 and split the sheath body 142 with the catheter tip 126; or pull the access guidewire 106 distally against a side of the sheath body 142 and split the sheath body 142 with the access guidewire 106 after initiation of the splitting of the sheath body 142 thereby. In another example, the splitting of the sheath body 142 by can be continued by the different sheath-splitting means such as by switching to withdraw the introducer needle 104 or the sheath body 142 by the finger tabs 144 and cut the sheath body 142 with the stationary or moveable blade 191 or 192; push the catheter tip 126 of the RICC 102 into the sheath body 142 and split the sheath body 142 with the catheter tip 126; or pull the access guidewire 106 distally against a side of the sheath body 142 and split the sheath body 142 with the access guidewire 106 after initiation of the splitting of the sheath body 142 by another one of the foregoing. In yet another example, the splitting of the sheath body 142 by can be continued by the different sheath-splitting means such as by switching to push the coupler 108 over the sheath body 142 and cut the sheath body 142 with the stationary or moveable blade 191 or 192 while holding the sheath body 142 in place in the needle tract after initiation of the splitting of the sheath body 142 by withdrawing the introducer needle 104 or the sheath body 142 by the finger tabs 144.

### Methods

Methods of the RICC insertion assembly 100 include a method for inserting the RICC 102 into a blood-vessel lumen of a patient. Such a method includes one or more steps selected from a RICC insertion assembly-obtaining step, a needle tract-establishing step, a blood-aspirating step, an access guidewire-advancing step, a sheath body-splitting step, a RICC-advancing step, an access guidewire-withdrawing step, a maneuver guidewire-advancing step, another RICC-advancing step, and a maneuver guidewire-withdrawing step.

The RICC insertion assembly-obtaining step includes obtaining the RICC insertion assembly 100. A set forth above, the RICC insertion assembly 100 includes the RICC 102, the introducer needle 104 including the sheath body 142 over the needle shaft 140, and the access guidewire 106 coupled together by the coupler 108. The proximal end of the access guidewire 106 is coupled to the extension arm 182 of the coupler 108 while the distal end of the access guidewire 106 is disposed in the introducer needle 104 by way of the valve module 180 of the coupler 108. Again, the proximal and distal ends of the access guidewire 106 enforce a loop in the access guidewire 106 as a result. The RICC 102 is disposed over the loop of the access guidewire 106 in the ready-to-deploy state of the RICC insertion assembly 100.

The needle tract-establishing step includes establishing a needle tract from an area of skin to the blood-vessel lumen with the introducer needle 104. Such a needle tract-establishing step can include flipping the extension arm 182, and, thus, the loop between two sides of the RICC insertion assembly 100 before puncturing the area of skin with the introducer needle 104. The needle tract-establishing step can also include ensuring blood flashback while establishing the needle tract.

Flipping the extension arm 182 and, thus, the loop between the two sides of the RICC insertion assembly 100 include flipping the loop between a sinistral side of the RICC insertion assembly 100 for a left-handed venipuncture and a dextral side of the RICC insertion assembly 100 for a right-handed venipuncture with the RICC insertion assembly 100. Indeed, flipping the loop from the sinistral side of the RICC insertion assembly 100 to the dextral side of the RICC insertion assembly 100 accommodates a left-handed venipuncture with the RICC insertion assembly 100. Likewise, flipping the loop from the dextral side of the RICC insertion assembly 100 to the sinistral side of the RICC insertion assembly 100 accommodates a right-handed venipuncture with the RICC insertion assembly 100.

Ensuring blood flashback while establishing the needle tract includes ensuring blood flashes back into the needle hub 145 of the introducer needle 104, the syringe tip 172 of the syringe 110 fluidly connected to the introducer needle 104, a barrel of the syringe 110, or a combination thereof. A slight vacuum can be drawn with the syringe 110 while establishing the needle tract such that the blood flashes back into at least the needle hub 145 of the introducer needle 104 upon establishing the needle tract. Ensuring the blood flashes back in accordance with the foregoing confirms the needle tract extends into the blood-vessel lumen.

The blood-aspirating step includes aspirating blood with the syringe 110 for confirmation the needle tract extends into the blood-vessel lumen before withdrawing the introducer needle 104 from the coupler 108 in the introducer needle-withdrawing step. Again, the sheath body 142 over the needle shaft 140 seals the needle slot 148 of the needle shaft 140 thereunder. In particular, the sheath body 142 seals the needle slot 148 outside of the valve module 180. The valve module 180, in turn, seals over the sheath opening 162 of the sheath body 142, which sheath opening 162 allows the access guidewire 106 to pass into the needle shaft 140 by way of the needle slot 148 in the ready-to-deploy state of the RICC insertion assembly 100. The valve module 180 also seals around the distal portion of the access guidewire 106. Such seals enable the syringe 110 to aspirate blood in the blood-aspirating step.

The access guidewire-advancing step includes advancing the distal end of the access guidewire 106 from its initial location in the needle shaft 140 just proximal of the needle tip 146 into the blood-vessel lumen, thereby securing blood-vessel access for the RICC 102 in the RICC-advancing step.

The sheath body-splitting step includes splitting the sheath body 142 away from the access guidewire 106, thereby allowing the access guidewire 106 to escape from the sheath body 142 and remain in the needle tract. The sheath body-splitting step can be initiated by can be initiated by withdrawing the introducer needle 104 or the sheath body 142 by the pair of finger tabs 144 extending from the sheath hub 143 around the proximal portion of the sheath body 142 to initiate cutting of the sheath body 142 with the stationary blade 191; twisting the introducer needle 104, the needle hub 145, or the sheath hub 143 around the proximal portion thereof to initiate cutting of the sheath body 142 with the moveable blade 192; triggering the one-or-more triggers disposed in the coupler housing 178 to initiate cutting of the sheath body 142 with the moveable blade 192; pushing the catheter tip 126 of the RICC 102 into the sheath body 142 to initiate splitting of the sheath body 142 with the catheter tip 126; or pulling the access guidewire 106 distally against the side of the sheath body 142 to initiate splitting of the sheath body 142 with the access guidewire 106. After the sheath body-splitting step is initiated, the sheath body-splitting step can be continued by a same or different sheath-splitting means than that used to initiate the sheath body-splitting step. As such, the sheath body-splitting step can be continued after initiating the sheath body-splitting step by pushing the coupler 108 over the sheath body 142 while holding the sheath body 142 in place to cut the sheath body 142 with the stationary or moveable blade 191 or 192; withdrawing the introducer needle 104 from the coupler 108 to cut the sheath body 142 with the stationary or moveable blade 191 or 192; withdrawing the sheath body 142 from the coupler 108 to cut the sheath body 142 with the stationary or moveable blade 191 or 192; pushing the catheter tip 126 of the RICC 102 further into the sheath body 142 with the advancing of the catheter tip 126 over the access guidewire 106 and into the blood-vessel lumen to split the sheath body 142 with the catheter tip 126; or pulling the access guidewire 106 distally toward the patient and against the side of the sheath body 142 to split the sheath body 142 with the access guidewire 106. Notably, an entirety of the introducer needle 104 including both the needle shaft 140 and the sheath body 142 is removed subsequent to sheath-splitting step, thereby leaving the access guidewire 106 in place in the blood-vessel lumen. When the sheath-splitting step simply involves withdrawing the introducer needle 104 from the coupler 108, whether in its entirety or piecemeal by way of the needle shaft 140 followed by the sheath body 142, the sheath-splitting step can be referred to as the introducer needle-withdrawing step.

The RICC-advancing step includes advancing the catheter tip 126 and, subsequently, the catheter tube 112 of the RICC 102 over the access guidewire 106 and into the blood-vessel lumen, thereby inserting the RICC 102 into the blood-vessel lumen.

The access guidewire-withdrawing step includes withdrawing the access guidewire 106 leaving the catheter tube 112 in place in the blood-vessel lumen.

The maneuver guidewire-advancing step includes advancing a maneuver guidewire into the blood-vessel lumen by way of the primary lumen 128 of the RICC 102 and to a lower ⅓ of an SVC of a heart of the patient.

The other RICC-advancing step includes advancing the distal portion of the catheter tube 112 farther into the blood-vessel lumen over the maneuver guidewire to the lower ⅓ of the SVC of the heart of the patient.

The maneuver guidewire-withdrawing step includes withdrawing the maneuver guidewire leaving the catheter tube 112 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A rapidly insertable central catheter (“RICC”) insertion assembly, comprising:
a RICC;
an introducer needle including:
a needle shaft including a longitudinal needle slot extending from a proximal portion of the needle shaft through a distal needle tip; and
a sheath body over the needle shaft sealing the needle slot thereunder except for that under a sheath opening to the needle slot in a proximal portion of the sheath body;
a coupler coupling the RICC and the introducer needle together, the coupler including:
a coupler housing; and
a valve module disposed in the coupler housing sealing the proximal portions of the needle shaft and the sheath body therein;
an access guidewire including:
a proximal end coupled to an extension arm of the coupler; and
a distal end disposed in the introducer needle; and
a sheath-splitting means for splitting the sheath body away from the access guidewire subsequent to a percutaneous puncture with the introducer needle and, thereby, allowing the access guidewire to escape from the sheath body and remain in a needle tract established by the percutaneous puncture, the splitting by the sheath-splitting means initiated by withdrawing the sheath body by a pair of finger tabs extending from a sheath hub around the proximal portion of the sheath body, twisting the introducer needle or a needle hub around the proximal portion of the needle shaft, triggering one or more triggers disposed in the coupler housing, or pushing a catheter tip of the RICC into the sheath body.

2. The RICC insertion assembly of claim 1, wherein the splitting by a same or different sheath-splitting means is continued after initiation by pushing the coupler over the sheath body while holding the sheath body in place.

3. The RICC insertion assembly of claim 1, wherein the splitting by a same or different sheath-splitting means is continued after initiation by withdrawing the introducer needle from the coupler.

4. The RICC insertion assembly of claim 1, wherein the splitting by a same or different sheath-splitting means is continued after initiation by withdrawing the sheath body from the coupler.

5. The RICC insertion assembly of claim 1, wherein the splitting by a same or different sheath-splitting means is continued after initiation by pushing the catheter tip of the RICC into the sheath body.

6. The RICC insertion assembly of claim 1, wherein the splitting by a same or different sheath-splitting means is continued after initiation by pulling the access guidewire distally against a side of the sheath body.

7. The RICC insertion assembly of claim 1, wherein the sheath-splitting means is a blade, the access guidewire, the catheter tip, or a combination thereof.

8. The RICC insertion assembly of claim 7, wherein the blade is disposed in the valve module under a distal end of the sheath opening, the blade including a distally facing blade edge configured to slide along the needle slot and cut the sheath body away from the access guidewire.

9. The RICC insertion assembly of claim 7, wherein the blade is spring-loaded on a compression spring, the compression spring configured to relax when the introducer needle or the needle hub is twisted or the one-or-more triggers disposed in the coupler housing are triggered.

10. The RICC insertion assembly of claim 1, wherein the proximal and distal ends of the access guidewire enforce a loop in the access guidewire, over which loop the RICC is disposed in a ready-to-deploy state of the RICC insertion assembly.

11. The RICC insertion assembly of claim 10, further comprising:
a syringe fluidly coupled to the introducer needle in the ready-to-deploy state of the RICC insertion assembly, the sheath body sealing the needle slot of the needle shaft thereunder outside of the valve module, the valve module sealing the sheath opening of the sheath body therein, and the valve module sealing around the access guidewire enabling the syringe to aspirate blood.

12. The RICC insertion assembly of claim 1, wherein the coupler housing includes a longitudinal coupler-housing slot configured to allow the access guidewire to escape from the coupler housing when the sheath body is split away from the access guidewire.

13. A method for inserting a rapidly insertable central catheter (“RICC”) into a blood-vessel lumen of a patient, comprising:
obtaining a RICC insertion assembly including the RICC, an introducer needle including a sheath body over a needle shaft, and an access guidewire coupled together by a coupler, a proximal end of the access guidewire coupled to an extension arm of the coupler and a distal end of the access guidewire disposed in the introducer needle by way of a valve module of the coupler;
establishing a needle tract from an area of skin to the blood-vessel lumen with the introducer needle;
advancing the distal end of the access guidewire from its initial location in the needle shaft just proximal of a needle tip of the needle shaft into the blood-vessel lumen; and
splitting the sheath body away from the access guidewire, thereby allowing the access guidewire to escape from the sheath body and remain in the needle tract, the splitting of the sheath body initiated by withdrawing the sheath body by a pair of finger tabs extending from a sheath hub around the proximal portion of the sheath body, twisting the introducer needle or a needle hub around a proximal portion of the needle shaft, triggering one or more triggers disposed a coupler housing of the coupler, or pushing a catheter tip of the RICC into the sheath body;
advancing the catheter tip of the RICC over the access guidewire and into the blood-vessel lumen, thereby inserting the RICC into the blood-vessel lumen.

14. The method of claim 13, wherein the splitting of the sheath body away from the access guidewire is continued after initiation by pushing the coupler over the sheath body while holding the sheath body in place.

15. The method of claim 14, wherein the needle shaft includes a longitudinal needle slot extending from a proximal portion of the needle shaft through the needle tip allowing the access guidewire to escape therefrom with the continued splitting of the sheath body away from the access guidewire.

16. The method of claim 13, wherein the splitting of the sheath body away from the access guidewire is continued after initiation by withdrawing the introducer needle form the coupler.

17. The method of claim 13, wherein the splitting of the sheath body away from the access guidewire is continued after initiation by withdrawing the sheath body from the coupler.

18. The method of claim 13, wherein the splitting of the sheath body away from the access guidewire is continued after initiation by pushing the catheter tip of the RICC further into the sheath body with the advancing of the catheter tip over the access guidewire and into the blood-vessel lumen.

19. The method of claim 13, wherein the splitting of the sheath body away from the access guidewire is continued after initiation by pulling the access guidewire distally toward the patient and against a side of the sheath body.

20. The method of claim 13, wherein the splitting of the sheath body away from the access guidewire is accomplished with a blade.

21. The method of claim 20, wherein the blade is disposed in the valve module under a distal end of the sheath opening, the blade including a distally facing blade edge configured to slide along the needle slot and cut the sheath body away from the access guidewire.

22. The method of claim 20, wherein the blade is spring-loaded on a compression spring, the compression spring configured to relax when the introducer needle or the needle hub is twisted or the one-or-more triggers disposed in the coupler housing are triggered.

23. The method of claim 13, further comprising withdrawing the access guidewire leaving the catheter tube in place in the blood-vessel lumen.
